(19) United States
(12) Patent Application Publication
(10) Pub. No.: US 2026/0341455 A1
(43) Pub. Date: Oct. 29, 2026

(54) INJECTION DEVICE AND ADD-ON DEVICE

(71) Applicant: Sanofi, Paris (FR)

(72) Inventors: Stefan Alt, Frankfurt am Main (DE); Michael Helmer, Frankfurt am Main (DE)

(21) Appl. No.: 19/107,382

(22) PCT Filed: Aug. 28, 2023
(86) PCT No.: PCT/EP2023/073459

§ 371 Date: Feb. 27, 2025

(30) Foreign Application Priority Data
Aug. 30, 2022 (EP) 22315202.6

(51) Int. Cl. G16H 20/17; A61M 5/20; A61M 5/24

(52) U.S. Cl. CPC G16H 20/17; A61M 5/20; A61M 5/24; A61M 2205/3523; A61M 2205/3569; A61M 2205/60

(57) ABSTRACT

Disclosed is an injection device for injecting of a dose of a medicament, the injection device including a housing having a body detachably connectable to a container part configured to accommodate a medicament container, a drive mechanism arranged in the body and operable to expel the dose of the medicament from the medicament container, a first antenna disposed on or in a first portion of the body and configured for wireless signal transmission with an electronic container identifier provided on or in the container part, a second antenna disposed on or in a second portion of the body electrically connected to the first antenna and configured for wireless signal transmission with a transceiver of an electronic module of an add-on device connectable to the injection device, and an electronic device identifier configured for wireless signal transmission with the transceiver.

### CROSS-REFERENCE TO RELATED APPLICATIONS

The present application is the national stage entry of International Patent Application No. PCT/EP2023/073459, filed on Aug. 28, 2023, and claims priority to European Application No. EP 22315202.6, filed on Aug. 30, 2022, the disclosures of which are incorporated herein by reference.

### TECHNICAL FIELD

The present disclosure relates to the field of injection devices, such as hand-held pen-type injectors. The disclosure further relates to add-on devices for such injection devices, in particular, to add-on devices attachable to a pen-type injector. In another aspect the disclosure relates to an injection system comprising an injection device and an add-on device. In further aspects the disclosure relates to a method of monitoring operation or use of an injection device.

### BACKGROUND

Drug delivery devices for setting and dispensing a single or multiple doses of a liquid medicament are as such well-known in the art. Generally, such devices have substantially a similar purpose as that of an ordinary syringe.

Drug delivery devices, such as pen-type injectors, have to meet a number of user-specific requirements. For instance, with patients suffering chronic diseases, such as diabetes, the patient may be physically infirm and may also have impaired vision. Suitable drug delivery devices especially intended for home medication therefore need to be robust in construction and should be easy to use. Furthermore, manipulation and general handling of the device and its components should be intelligible and easy understandable. Such injection devices should provide setting and subsequent dispensing of a dose of a medicament of equal or variable size. Moreover, a dose setting as well as a dose dispensing procedure must be easy to operate and has to be unambiguous.

A patient suffering from a particular disease may require a certain amount of a medicament to either be injected via a pen-type injection syringe.

Some drug delivery or injection devices provide selecting of a dose of a medicament of variable size and injecting a dose previously set. Other injection devices provide setting and dispensing of a fixed dose. Here, the amount of medicament that should be injected in accordance to a given prescription schedule is always the same and does not change or cannot be changed over time.

Some injection devices are implemented as reusable injection devices offering a user to replace a medicament container, such as a cartridge. Other injection devices are implemented as a disposable injection device. With disposable injection devices it is intended to discard the entirety of the injection device when the content, i.e. the medicament, has been used up.

In order to control and to supervise administering of medication conducted by users or patients themselves it is beneficial to assist the user by making use of an external electronic device, such as a mobile electronic device, e.g. implemented as a smartphone, a tablet computer, or a smart watch. A software application provided on such external electronic devices may interact with the user and may provide instructions or recommendations to the user of how to correctly use the injection device.

In order to control and to supervise administering of medication conducted by users or patients themselves it is desirable to provide an automated detecting and logging of a repeated and regular use of the drug delivery device. A rather automated recording of doses injected by a user would offer a significant advantage over a manual dose logging in terms of security and convenience.

There exist numerous add-on devices configured for use with injection devices that offer an electronic detection and monitoring of repeated dose injection procedures.

Typically, such add-on devices or auxiliary devices can be detachably connected to an injection device. An add-on device typically comprises a detector or detector arrangement operable to detect a date and/or time when the user sets or injects a dose of the medicament. Some add-on devices also provide a quantitative measurement of a size of a dose currently set or dispensed. Some add-on devices are intended for use with a series of injection devices. This may particularly apply with disposable injection devices, that are intended to become discarded after use or after the medicament located therein has been used up.

With reusable injection devices, e.g. of pen-injector type a container part containing the medicament container may be detachably connected to a body of the injection device, wherein the body comprises a drive mechanism operably engageable with the medicament container for expelling or withdrawing a dose of the medicament from the medicament container.

Some injection devices of reusable type may be operable for setting and dispensing of different medicaments, e.g. provided in different container parts and/or different medicament containers. Here, a specific container part and/or medicament container should only be used with a dedicated drive mechanism and/or body of an injection device. Hence, a correct pairing of a medicament container with a suitable drive mechanism has to be ensured.

Also, for manually actuated injection devices, such as pen-type injectors, the expelled dose volume per settable dose increment is usually determined and fixed by the device mechanics of the drive mechanism. Therefore, reusable injection devices are commonly dedicated to a single drug to avoid incorrect dosing. However, some drug type, like insulin, are provided in common unit/volume rations (e.g. U100, U300), so a reusable pen could potentially support multiple drugs of the same group. As this increases the risk of potentially dangerous mix-ups, there should be provided a technical solution to provide an enhanced mix-up prevention.

It is hence desirable to improve patient safety for reusable injection devices of e.g. pen-injector type. The improvements should help to prevent unintended mix-up of medicament containers and drive mechanisms for such injection devices. Furthermore, it is desirable to facilitate and/or to simplify recording and monitoring of doses set and/or injected by such injection devices.

### SUMMARY

In one aspect there is provided an injection device for injecting of a dose of a medicament. The injection device comprises a housing comprising a body detachably connectable to a container part. The container part is configured to accommodate a medicament container. The injection device further comprises a drive mechanism arranged in the body and operable to expel and/or to withdraw the dose of the medicament from the medicament container.

The injection device further comprises a first antenna on or in a first portion of the body. The antenna is configured for wireless signal transmission with an electronic container identifier provided on or in the container part. The injection device further comprises a second antenna on or in a second portion of the body. The second antenna is electrically connected to the first antenna and is further configured for wireless signal transmission with a transceiver of an electronic module of an add-on device connectable to the injection device.

The injection device further comprises an electronic device identifier on or inside the body and configured for wireless signal transmission with the transceiver of the electronic module of the add-on device.

With some examples the first antenna and the second antenna are separated from each other, e.g. along a longitudinal direction of the injection device. The injection device may be implemented as a mechanically-implemented injection device, where a user has to exert a dispensing force required for expelling the medicament from the medicament container. Apart from the first antenna, the second antenna and the electronic device identifier the injection device may be void of any further electric or electronic components.

The second antenna is particularly designed and configured to wirelessly communicate with the add-on device when the add-on device is attached or connected to the injection device. The electrical connection between the first antenna and the second antenna allows and supports a signal transmission between the first antenna and the add-on device even in situations in which the wireless transmission range of the add-on device is shorter than the distance between the add-on device and the first antenna when the add-on device is suitable fixed to the injection device. Here, the electrical connection between the first antenna and the second antenna provides a kind of a relay or spatial range extender function. The add-on device is operable to communicate and/or is configured for signal transmission with the first antenna via the second antenna and by the electric connection between the first antenna and the second antenna.

The electronic identifier is likewise configured for wireless signal transmission with the transceiver. The add-on device and hence its transceiver is operable to communicate with both, the electronic device identifier and the second antenna as provided on or inside the injection device. This way, the present injection device provides a twofold communication or signal transmission with an external electronic device. When the add-on device is assembled or connected to the injection device the electronic device identifier is operable to communicate with the add-on device. Furthermore and through the electrical connection between the first and the second antennas there is also enabled a separate or additional communication between the electronic container identified and the add-on device. This signal transmission is provided through a communication link formed or established between electronic container identifier and the first antenna, the electrical connection between the first antenna and the second antenna and by way of the wireless signal transmission between the second antenna and the add-on device.

By way of the electronic identifier on or inside the body of the injection device, the first and the second antennas and their mutual electrical interconnection there can be provided a twofold communication link with the add-on device. The add-on device may become operable to read data or information from the electronic container identifier as well as to read or to obtain information from the electronic device identifier. This way, the add-on device will be enabled to read or to obtain information from both, the container part as well as from the body of the injection device, thus allowing to conduct a comparison and a checkup if the data being indicative of the medicament container matches with the data being indicative of the drive mechanism or body of the injection device.

In this way there can be provided a rather simplified reading or data gathering mechanism for providing the relevant information to the add-on device when the add-on device is attached to e.g. a proximal end of the injection device and while the electronic container identifier is outside the transmission range of the add-on device.

Reading of the electronic container identifier is thus enabled through the electrical connection between first and second antennas of the injection device. Concurrently, the add-on device is operable to wirelessly read or to wirelessly obtain information or data from the electronic device identifier.

This way, all electronically readable information as provided in or on the injection device, namely information or data as provided by the electronic container identifier and as provided by or in the electronic device identifier can be provided to the add-on device wirelessly with the add-on device being attached to a dedicated portion of the injection device, e.g. while being attached at or near the proximal end of the injection device.

According to a further example the injection device comprises an electric transmission line interconnecting the first antenna and the second antenna. The transmission line is configured to transmit electrical signals between the first and the second antennas. This way there can be provided an indirect signal transmission between the transceiver of the electronic module of the add-on device with the first antenna even when the first antenna is out of the transmission range of the transceiver of the add-on device.

According to a further example the transmission line also provides electrical power between the first and the second antennas. The transmission line may be provided as a power line operable to transfer and/or to provide electrical energy or electrical power from the second antenna to the first antenna. The second antenna may be inductively coupled with the transceiver of the add-on device when the add-on device is attached and/or connected to the injection device. This way, the electrical power induced by the add-on device into the second antenna can be transferred via the transmission line to the first antenna and can be used to wirelessly obtain or to wirelessly read respective information or data from the electronic container identifier, which is within the transmission range of the first antenna.

This way, at least one or both identifiers, namely the electronic container identifier as well as the electric device identifier could be implemented as passive radiofrequency transmission devices, e.g. as a passive RFID tags, NFC tag or UWB tag.

With some examples, the electric transmission line connected with the first antenna and connected with the second antenna forms or constitutes a bridge antenna, such as a NFC bridge antenna or RFID bridge antenna. Thus, the bridge antenna includes the first antenna and the second antenna spaced apart from one another and coupled together by the electric transmission line, e.g. by an electric conductor.

Typically, the first antenna is located proximate to the electronic container identifier, which may be implemented as a passive RFID or passive NFC tag. The second antenna is located proximate to the transceiver of the electronic module of the add-on device when the add-on device is connected to the injection device. Here, the transceiver forms or constitutes a RFID reader.

An electromagnetic signal transmitted by the transceiver is received by the second antenna and is transmitted via the electric transmission line to the second antenna, which then communicates with the electronic container identifier provided on or in the container part. A response from the electronic container identifier may be then retransmitted via the first antenna to the second antenna and further to the transceiver of the electronic module of the add-on device.

This way, the first antenna and the second antenna, which are mutually interconnected by the transmission line, provide a wired range extension for the wireless signal transmission between the electronic container identifier and the transceiver of the add-on device.

With some examples, the first antenna is matched or tuned to a respective antenna of the electronic container identifier. Accordingly, the second antenna may be matched or tuned to the transceiver of the electronic module of the add-on device.

The first antenna and the second antenna may be mutually tuned to a common transmission frequency.

The electric transmission line interconnecting the first and the second antennas may be of passive type. It may provide a wired connection between the first and the second antennas. The transmission range of the first antenna is extended through the longitudinal extension of the transmission line and the position of the second antenna relative to the first antenna; and vice versa.

With a further example the electronic device identifier comprises an integrated device circuit with a device memory configured to store at least one of a device information and a use related data. In the same or in a similar way also the electronic container identifier may comprise an integrated circuit with a memory configured to store at least one of a container information and a use-related data.

The use-related data may be indicative of a status of use of the injection device. The status of use may be determinable by the add-on device. The status of use may contain information such as: total number of doses set or injected, a total amount of medicament injected by the injection device, a type, a name and/or concentration of medicament injected by the injection device, a LOT number of a medicament container and other medicament-or medicament container-related information that may be useful for the actual or future use of the injection device.

The container information may be indicative of at least one of a type of a medicament contained in the medicament container a concentration of a pharmaceutical substance of the medicament contained in the container, a manufacturing date of the medicament, a use by date of the medicament or, a LOT number of the medicament or medicament container and/or environmental parameters, such as temperature, humidity or radiation intensity to which the medicament container has been exposed.

The device information may comprise information or data with regard to the type of the injection device. The device information may be specific about a type of an injection mechanism or drive mechanism of the injection device. The device information may thus characterize the dose setting and/or dose dispensing operability of the respective injection device. The device identification may further contain data or information about suitable medicaments or medicament containers to be exclusively used with the respective injection device.

According to a further example of the injection device and for wireless signal transmission between the integrated device circuit and the transceiver the integrated device circuit is electrically connected to the second antenna or the integrated device circuit is electrically connected to an identifier antenna of the electronic device identifier. The identifier antenna is configured for wireless signal transmission with the transceiver. This way the electronic device identifier is either equipped with an own and separate identifier antenna or the second antenna of the injection device is connected with the integrated device circuit in order to enable wireless signal transmission between the add-on device and the integrated device circuit of the electronic device identifier.

With some examples, wherein the electronic device identifier is void of an own identifier antenna the add-on device is typically provided with a wireless transmission collision handling function so as to distinguish between signal transmission with regards to the electronic container identifier by the first and the second antennas and signal transmission with the electronic device identifier, which takes place via the second antenna.

With the second antenna being electrically connected to the integrated device circuit and wherein the second antenna provides an antenna for the electronic device identifier the injection device must be only provided with the first antenna and with the second antenna. Here, the second antenna provides a wireless signal transmission between the add-on device and the electronic container identifier via the first antenna. The second antenna further provides signal transmission between the add-on device and the electronic device identifier. With this twofold transmission function of the second antenna construction space for a separate antenna of the electronic device identifier can be saved.

Moreover, costs and expenditure for the production of the electronic device identifier can be reduced and the integration or assembly of the second device identifier on or inside the injection device can be simplified.

Here and when the second antenna is electrically connected to the integrated device circuit and/or when the second antenna provides an antenna for the electronic device identifier the transceiver of the electronic module is typically equipped or provided with a collision management functionality. It may be operable to conduct or to control a temporal multiplexing to avoid crosstalk between signals received from the electronic device identifier and the electronic container identifier.

Moreover, the transceiver of the electronic module may be operable to communicate with the electronic container identifier via the first and second antennas across the electronic transmission line at a first time or time interval and may be further operable to communicate with the electronic device identifier at a second time or time interval, wherein first and second times or first and second time intervals do not overlap.

With other examples, wherein the integrated device circuit of the electronic device identifier is equipped with an own identifier antenna there may be provided an easier separation of signal transmission with regards to the electronic container identifier and the electronic device identifier. Here, the second antenna may be exclusively used for communication and signal transmission with the electronic container identifier via the first antenna.

According to a further example the body of the injection device comprises a distal end connectable to the container part. The body further comprises a proximal end opposite the distal end. The first antenna is arranged at or near the distal end. The second antenna is arranged at or near the proximal end of the body. The longitudinal distance between the first and the second antennas may be larger than the transmission range of the transceiver of the electronic module of the add-on device. Signal transmission between the second antenna and the transceiver of the add-on device may be based on a near field communication protocol. The transmission range may be in a range of only a few centimeters. Through the wired connection between the first and the second antennas on or inside the body of the injection device any signals wirelessly transmitted between the second antenna and the add-on device can be equally shared and/or transmitted to the first antenna, thus enabling a wireless reading of the electronic container identifier, being within a transmission range of the first antenna.

According to a further example the injection device also comprises the container part. The electronic container identifier is arranged on or in one of the container part and the medicament container accommodated by the container part. The container part may be implemented as a cartridge holder configured for detachably connecting with a portion of the body. Typically, a proximal end of the container part is detachably connectable with a distal end of the body. With some examples the medicament container comprises or constitutes a cartridge filled with a liquid medicament. The medicament container is typically arranged inside the container part. The container part and the medicament container may be preassembled and may be mutually fixed. The medicament container inside the container part may constitute or form a container assembly of the injection device.

The electronic container identifier may be provided at or near a proximal end of the container part. With some examples the container identifier is provided on or inside the medicament container. With some examples the electronic container identifier is provided on an outside surface or inside surface of a sidewall of the container part. It may be also provided on an outside surface of the medicament container. It may be provided on an outside surface of a tubular-shaped barrel of the medicament container or cartridge. With further examples the electronic container identifier may be provided on or inside a stopper of the medicament container, which stopper is longitudinally displaceable inside the barrel of the medicament container in order to expel or to withdraw the dose of the medicament from the medicament container.

With some examples the electronic container identifier is provided on or inside a label adhered to one of the container part of the injection device and the medicament container to be arranged inside the container part.

According to a further example the electronic container identifier comprises a container antenna and an integrated container circuit connected to the container antenna. The integrated container circuit comprises a container memory configured to store at least one of the container information and the use-related data.

With a further example the container memory is readable by the transceiver of the electronic module of the add-on device when attached to the injection device. Reading of the container memory is provided through wireless signal transmission between the first antenna and the container antenna and through wireless signal transmission between the second antenna and the transceiver of the add-on device. Signal transmission between the first antenna and the second antenna is provided by the electrical connection therebetween. Typically, signal transmission between the first antenna and the second antenna is provided by the electric transmission line interconnecting the first antenna and the second antenna.

According to a further example at least one of the electronic container identifier and the electronic device identifier comprises a passive RFID, a passive NFC tag or a passive UWB action. Implementation of passive RF communication tags for the electronic container identifier helps to reduce manufacturing costs and manufacturing expenditures.

With a further example the drive mechanism comprises at least one movable component, which is subject to a movement relative to the housing or medicament container during at least one of setting of the dose and injecting of the dose of the medicament. With some examples the movable component is one of a dose dial being rotatable relative to the housing for setting of a dose, a dial sleeve rotatable relative to the housing during setting of the those or dispensing of the dose, a drive sleeve or a drive member, which is subject to a rotational and/or longitudinal motion relative to the housing during at least one of setting of the dose and dispensing of the dose. With some examples the movable component is a clicker or clicking element operable to generate an audible sound recordable by the sensor of the add-on device. With further examples the movable component is a piston rod operably engaged with a piston of the medicament container for displacing the piston relative to the medicament container during injecting of the dose. With further examples the movable component is a single dose indicating member, whose movement and/or position relative to the housing or device body is indicative of the size of a dose currently set. With further examples the movable component is a last dose member, whose position relative to the housing of the injection device is directly indicative of the residual amount of medicament left in the medicament container or cartridge.

According to another aspect the present disclosure further relates to an add-on device for attaching to an injection device. The add-on device comprises a device body fastenable to a portion of the injection device. The add-on device comprises an electronic module comprising a module processor and a transceiver. The electronic module is operable to read data from the electronic container identifier by the first antenna and by the second antenna and the electronic module is further operable to read the data from the electronic device identifier wirelessly.

With some examples the add-on device is configured for attachment and configured for interaction with an injection device as described above. Insofar, all features, effects and benefits as described above in connection with the injection device equally apply to the add-on device; and vice versa.

With some examples and when appropriately attached to the injection device the add-on device is operable to obtain data from both, the electronic device identifier and from the electronic container identifier. In this way, there can be provided device information being indicative of the injection device as well as container information being indicative of the medicament container. The add-on device may be further operable to process the respective data or information in order to determine if the medicament container as identified through wireless signal transmission with the electronic container identifier matches the drive mechanism or body of the injection device, wherein respective information or data of the drive mechanism is obtainable by wirelessly reading the electronic device identification.

According to a further example the transceiver of the electronic module is configured for wireless signal transmission with the second antenna when the device body is fastened to the injection device. Wireless signal transmission with the second antenna may also provide a wireless signal transmission between the first antenna and the electronic container identifier being within the transmission range of the first antenna. Signal transmission between the first and the second antennas may also include power transmission between the first and the second antennas, wherein the electrical power for e.g. reading of the electronic container identifier may be provided by a reader, e.g. implemented in form of the transceiver of the electronic module of the add-on device. Here, both wireless communication tags of the electronic container identifier and of the electronic device identifier may be implemented as passive communication tags.

According to a further example the transceiver of the electronic module is configured for wireless signal transmission with an identifier antenna of the electronic device identifier when the device body is fastened to the injection device. Here, the transceiver may be operable to communicate with at least two different antennas as provided on or inside the injection device. The transceiver of the add-on device may be operable to wirelessly communicate with the second antenna as well as to wirelessly communicate with the separate identifier antenna of the electronic device identifier.

Having two separate antennas for wireless signal transmission with the electronic container identifier and with the electronic device identifier, respectively, may be beneficial for separate signal transmission, which due to different antenna transmission characteristics may be void of any crosstalk.

According to a further example of the add-on device the electronic module is operable to separate and/or to distinguish between signals received from the electronic container identifier and the electronic device identifier. For this, the electronic module may be equipped with a wireless transmission collision handling function. It may provide a to distinguish between signals obtained from the electronic container identifier and signals obtained from the electronic device identifier. This way, the electronic module may distinguish signals provided by and received from the electronic container identifier from signals provided by and received from the electronic device identifier.

According to a further example the electronic module is operable to induce and to transmit electric power to the first antenna via the second antenna and the transmission line electrically connecting the first antenna and the second antenna. The electric power is effective to induce signal transmission between the first antenna and the electronic container identifier when the electronic container identifier is within the transmission range of the first antenna. This way the electronic container identifier may be implemented as a passive radiofrequency communication tag, thus allowing to simplify the structure of the electronic container identifier and/or to reduce respective manufacturing costs.

With a further example the add-on device comprises a sensor operable to quantitatively determine at least one of a position and a movement of a movable component relative to at least one of the housing, the medicament container and the device body. The sensor is operable to generate a respective sensor signal. The module processor of the electronic module of the add-on device is further operable to determine or to calculate a size of the dose of the medicament, which is currently set or dispensed by the injection device. Determination or calculation of the dose size is conducted on the basis of the sensor signal and on the basis of data received from one of the electronic container identifier and the electronic device identifier.

With some examples the module processor is further operable to determine or to calculate the size of the dose not only on the basis of the sensor signal(s) but also on the basis of at least one of the container information and the device information as obtained from the machine-readable identification.

With the present add-on device, and by the above described acquisition of at least one of a device information and a container information a somewhat automated calibration of the dose size determination or dose size calculation can be provided. Hence, the add-on device itself is capable to derive and/or to process medicament container specific information and/or device specific information, e.g. in the course of assembly to the injection device. In response to obtain the container information and/or the device information the add-on device may be configured to conduct a calibration routine. This way the measurable sensor signals as obtained by the sensor of the add-on device can be translated or re-calculated into correct dose size information.

According to a further example the electronic module of the add-on device is operable to read at least one of a device information and a use-related data from the electronic device identifier. The electronic module is further operable to read at least one of a container information and a use-related data from the electronic container identifier. This way, the electronic module may obtain both, device-related information as well as container related information being characteristic of the drive mechanism and being characteristic of the medicament container medicament contained therein.

The electronic module may be further operable to process respective device information and container information. The electronic module may be particularly operable to compare the device information with the container information in order to reveal if the device information matches the container information and/or to assert that the container part and/or the medicament container and hence the medicament contained therein matches with the body and/or with the drive mechanism of the injection device.

According to a further example the electronic module is operable to compare the device information with the container information and is further operable to determine, if the device information matches with the container information.

According to a further example the electronic module is operable to activate a locking mechanism of the drive mechanism when the container information does not match the device information. Here, the locking mechanism is operable to block at least one of setting of the dose and dispensing of the dose. For this, the electronic module may comprise a locking controller operably engageable with the locking mechanism of the drive mechanism of the injection device when the add-on device is correctly assembled to the injection device.

With some examples the electronic module may be operable to keep the locking mechanism of the drive mechanism locked per default until a matching combination of container information and device information is obtained by the electronic module of the add-on device.

Here, the locking mechanism of the drive mechanism may be also in a locking configuration per default. It may be unlocked or unlockable only and exclusively by the locking controller of the add-on device. Unlocking of the locking mechanism of the drive mechanism of the injection device may thus require a correct assembly of the add-on device to the injection device as well as acquisition and reading of matching data with regards to device information as provided by the electronic device identifier and container information as provided by the electronic container identifier. Both information or data, i.e. the device information and the container information can be acquired by a single transceiver of the add-on device when the add-on device is e.g. attached to proximal end of the injection device.

With a further aspect the present disclosure also relates to an injection system comprising an injection device as described above and further comprising an add-on device as described above. The injection system may optionally comprise at least one or several external electronic devices, such as a smartphone, a smartwatch and a tablet computer or some other electronic device that is capable to communicate with the add-on device.

Furthermore and according to another aspect the present disclosure also relates to a method of monitoring operation of an injection device. The method comprises the steps of attaching an add-on device as described above to an injection device. Typically, the add-on device as described above is connected to an injection device as described above. In a subsequent step device information is obtained or read from the electronic body identifier by the electronic module of the add-on device. In a further step container information is read or obtained from the electronic container identifier by the electronic module of the add-on device via the first and the second antennas of the injection device.

Thereafter and in a further step the device information and the container information obtained by reading of the electronic body identifier and by reading of the electronic container identifier is evaluated. The respective information may be compared with each other and/or may be compared with predefined data e.g. stored in a module memory of the electronic module of the add-on device.

By comparing or evaluating the device information and the container information it can be checked if the medicament container as identified or characterized by the electronic container identifier matches the body or drive mechanism of the injection device as identified or characterized by the electronic device identifier.

Obtaining of the device information and the container information may not require any user interaction. With some examples and upon installing the add-on device to the dedicated portion of the injection device the add-on device may be configured to autonomously conduct respective reading procedures in order to obtain the device information and the container information wirelessly.

The add-on device may not only limited to detect a matching between the type of a medicament container with a body or drive mechanism of an injection device. Reading and evaluating of device information and container information may also provide an effective counterfeiting drug prevention. It may be checked if the medicament as provided inside the medicament container has not expired. Moreover, it may be also checked if and in how far the medicament container has been stored and/or transported within a predefined temperature range.

Respective device information and/or container information can be stored and updated in the memory of the integrated circuit of at least one or both of the electronic container identifier and the electronic device identifier.

Insofar and generally, the electronic module and/or the module processor may be operable to process any of the device information, the use-related data and the container information so as to check if the respective information mutually match and represent an allowable and intended use or configuration of the injection device.

According to a further aspect the present disclosure also relates to a computer program, e.g. a computer program product comprising computer readable instructions, which when executed by a module processor of an add-on device as described above cause the module processor to conduct the steps of the method as described above.

The computer program and its respective computer readable instructions are particularly operable to read the device information from the electronic body identifier, to read the container information from the electronic container identifier and to evaluate the device information and the container information, e.g. to conduct a comparison or assertion if done device information matches with the container information.

Typically, the computer program is executable by an add-on device as described above. It is further operable to conduct a method of monitoring operation of the injection device with the add-on device as described above. Insofar, the computer program and its computer readable instructions are operable to cause a processor of the add-on device to conduct a method of monitoring or checking the injection device as described above. The computer readable instructions may be executable by a processor of an add-on device as described above.

Additionally, or alternatively and when an external electronic device of an injection system as described above is configured to read-out the machine-readable identification of the injection device the computer program and its computer readable instructions may be at least in part executable by a processor of the external electronic device.

The computer program is to be executed by a processor of an add-on device as described above in order to monitor and/or record operation of the injection device when the add-on device is attached thereto. Insofar, all features, effects and benefits as described above in connection with the add-on device and/or described in connection with the method of configuring the add-on device as well as any effects, features and benefits as described above in connection with the injection system equally apply to the computer program; and vice versa.

The present disclosure further discloses and proposes a computer program including computer-executable instructions for performing the method according to the disclosed method/device / system in one or more of the examples enclosed herein when the program is executed on a processor, computer or computer network. Specifically, the computer program may be stored on a computer-readable data carrier. Thus, specifically, one, more than one or even all of the method steps as indicated above may be performed by using a computer or a computer network, typically by using a computer program.

The present disclosure further discloses and proposes a computer program product having program code means, in order to perform the method according to the disclosed method / system in one or more of the embodiments enclosed herein when the program is executed on a computer or computer network. Specifically, the program code means may be stored on a computer-readable data carrier.

Further, the present disclosure discloses and proposes a data carrier having a data structure stored thereon, which, after loading into a processor, computer or computer network, such as into a working memory or main memory of the processor, computer or computer network, may execute the method according to one or more of the examples disclosed herein.

The present disclosure further proposes and discloses a computer program product with program code means stored on a machine-readable carrier, in order to perform the method or parts thereof according to one or more of the examples disclosed herein, when the program is executed on a processor, computer or computer network. As used herein, a computer program product refers to the program as a tradable product. The product may generally exist in an arbitrary format, such as in a paper format, or on a computer-readable data carrier. Specifically, the computer program product may be distributed over a data network.

With a further example the injection device comprises a dose dial and a trigger, e.g. provided at a proximal longitudinal end of the injection device. The injection device may be implemented as a pen-type injector. The dose dial and/or the trigger may be provided at a proximal longitudinal end of the pen-type injector. Here, the machine-readable identification may be provided in or on the dose dial and/or the trigger. The add-on device may be configured for releasable or detachable fastening to the proximal end of the injection device.

The add-on device, in particular its device body may comprise a receptacle to fit onto the dose dial and/or trigger. By implementing or arranging the machine-readable identification in or on the trigger or dose dial a geometric distance between the machine-readable identification and a reader or transceiver of the add-on device for readout of the machine-readable identification can be reduced to a minimum thus allowing to implement the transceiver or reader of the add-on device as a wireless near field transceiver operable to read a respective near field communication tag of the machine-readable identification of the device. This way, readout of the machine-readable identifier can only take place when the add-on device is correctly mounted to the proximal end of the injection device.

According to a further example the injection system comprises an external electronic device, e.g. implemented as a smartphone, a smart watch or as a tablet computer. The external electronic device may be operable to conduct at least one or several steps of the method of monitoring operation of the injection device.

Generally, the scope of the present disclosure is defined by the content of the claims. The disclosure is not limited to specific embodiments or examples but comprises any combination of elements of different embodiments or examples. Insofar, the present disclosure covers any combination of claims and any technically feasible combination of the features disclosed in connection with different examples or embodiments.

In the present context the term ‘distal’ or ‘distal end’ relates to an end of the injection device that faces towards an injection site of a person or of an animal. The term ‘proximal’ or ‘proximal end’ relates to an opposite end of the injection device, which is furthest away from an injection site of a person or of an animal.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short-or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20 °C), or refrigerated temperatures (e.g., from about 4 °C to about 4 °C). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide.

Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Examples of insulin derivatives are, for example, B29-N-myristoyl-des(B30) human insulin, Lys(B29) (N-tetradecanoyl)-des(B30) human insulin (insulin detemir, Levemir®); B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-gamma-glutamyl)-des(B30) human insulin, B29-N-omega-carboxypentadecanoyl-gamma-L-glutamyl-des(B30) human insulin (insulin degludec, Tresiba®); B29-N-(N-lithocholyl-gamma-glutamyl)-des(B30) human insulin; B29-N-(ω-carboxyheptadecanoyl)-des(B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091, MAR-701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrom. Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present invention include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present invention, which encompass such modifications and any and all equivalents thereof.

An example drug delivery device may involve a needle-based injection system as described in Table 1 of section 5.2 of ISO 11608-1: 2014(E). As described in ISO 11608-1: 2014(E), needle-based injection systems may be broadly distinguished into multi-dose container systems and single-dose (with partial or full evacuation) container systems. The container may be a replaceable container or an integrated non-replaceable container.

As further described in ISO 11608-1: 2014(E), a multi-dose container system may involve a needle-based injection device with a replaceable container. In such a system, each container holds multiple doses, the size of which may be fixed or variable (pre-set by the user). Another multi-dose container system may involve a needle-based injection device with an integrated non-replaceable container. In such a system, each container holds multiple doses, the size of which may be fixed or variable (pre-set by the user).

As further described in ISO 11608-1: 2014(E), a single-dose container system may involve a needle-based injection device with a replaceable container. In one example for such a system, each container holds a single dose, whereby the entire deliverable volume is expelled (full evacuation). In a further example, each container holds a single dose, whereby a portion of the deliverable volume is expelled (partial evacuation). As also described in ISO 11608-1: 2014(E), a single-dose container system may involve a needle-based injection device with an integrated non-replaceable container. In one example for such a system, each container holds a single dose, whereby the entire deliverable volume is expelled (full evacuation). In a further example, each container holds a single dose, whereby a portion of the deliverable volume is expelled (partial evacuation).

### BRIEF DESCRIPTION OF THE DRAWINGS

In the following, numerous examples of a data logging device for monitoring use of an injection device as well as a respective injection device will be described in greater detail by making reference to the drawings, in which:

FIG. 1 schematically illustrates an example of an injection device,

FIG. 2 shows the process of assembling an add-on device to a proximal end of the injection device,

FIG. 3 shows an injection device in a longitudinal schematic cross-section,

FIG. 4 shows a configuration of an injection system including the injection device, the add-on device and at least one external electronic device,

FIG. 5 shows an example of an add-on device before assembling to the injection device,

FIG. 6 shows the add-on device of FIG. 5 when attached to the injection device,

FIG. 7 shows a block diagram of one example of the injection device and the add-on device.

FIG. 8 shows the example of FIG. 7 when the add-on device is attached to the injection device,

FIG. 9 shows another example of an injection device,

FIG. 10 is illustrative of a cross-section through the injection device of FIG. 7 or 8,

FIG. 11 is illustrative of a cross-section through the injection device according to FIG. 9.

FIG. 12 shows a block diagram of the electronic circuit of the machine-readable identification,

FIG. 13 shows a block diagram of one example of the injection device with the add-on device attached thereto,

FIG. 14 shows a block diagram of another example of the injection device with the add-on device attached thereto,

FIG. 15 shows a block diagram of the add-on device, the injection device and the external electronic device,

FIG. 16 shows an example of the electronic circuit on a substrate,

FIG. 17 shows a flowchart of a method of configuring the add-on device.

### DETAILED DESCRIPTION

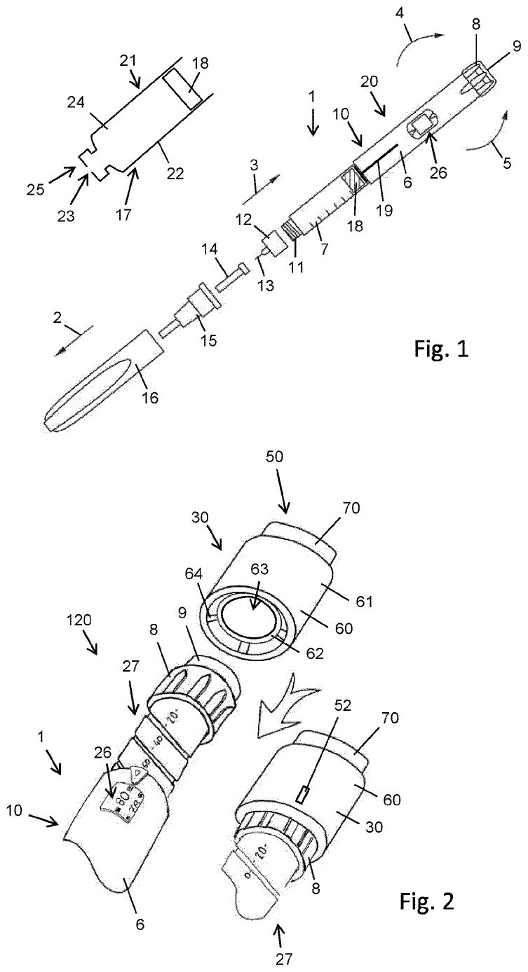

FIG. 1 shows an example of a drug delivery device, which is implemented as a handheld injection device 1. The injection device 1 may comprise or may be implemented as a pen-type injector. It may be implemented as a disposable injection device or as a reusable injection device. With some examples the injection device 1 is implemented as an autoinjector. The injection device 1 is of elongated shape. It may extend along a longitudinal direction. Towards a longitudinal distal direction 2 the drug delivery device 1 comprises a dispensing end for dispensing or injecting the medicament 24. Towards the proximal direction 3 the injection device 1 comprises at least one of a dose member 8 and a trigger 9, by way of which a dose of equal or individual or different size can be set and dispensed, respectively.

The injection device 1 comprises a housing 10. The housing 10 may comprise numerous housing components, such as a body 6 and a container part 7, e.g. implemented as a cartridge holder 7. The body 6 may be sized and configured to accommodate a drive mechanism 20. The container part 7 is sized and configured to accommodate a medicament container 21, e.g. implemented as a cartridge containing the liquid medicament 24. The medicament container 21 comprises a tubular-shaped barrel 22 sealed towards the distal end by a seal 23. The seal 23 may comprise a pierceable septum fixed to an outlet 25 of the medicament container 21.

Towards a proximal end the interior of the barrel 22 is sealed by a piston 18 or stopper, which is slidably disposed inside the barrel 22.

By advancing the piston 18 towards the distal direction 2 a dose of the medicament 24 can be expelled from the medicament container 21. In use the medicament container 21 is arranged inside the container part 7. The drive mechanism 20 of the injection device 1 comprises a piston rod 19, which is displaceable in distal direction 2 for advancing the piston 18 towards the outlet 25 of the medicament container 21. Details of the drive mechanism are not further illustrated and described here. With some examples, the drive mechanism 20 may be implemented as an all-mechanical drive mechanism, where a user has to provide an entirety of a dispensing force required to move the piston rod 19 and hence the piston 18 in distal direction 2. With other examples, the drive mechanism comprises a mechanical energy storage configured to provide at least a portion of the dispensing force. Examples of drive mechanisms can be found e.g. in WO 2004/078241 A1, WO 2014/033197 A1 or WO 2014/033195 A1 the entirety of which are herein incorporated by reference.

With some examples, as e.g. described or shown in FIG. 2 the injection device 1 and hence the drive mechanism 20 may comprise a dial extension 27, which projects and moves in proximal direction 3 from a proximal end of the body 6 when or during setting a dose and which returns into its initial distal end position during a dose injection procedure. For this, a user may use a thumb 114 of his hand 110 to exert a distally directed pressure onto the trigger 9 thereby urging the dial extension 27 in distal direction 2 during and/or for a dose injection procedure.

For setting or dialing of a dose a user may twist or rotate the dose dial 8, e.g. in a dose incrementing direction 4, hence in a clockwise sense as seen from the proximal end. For correcting a dose previously set the user may also rotate the dose dial 8 in an opposite dose decrementing direction 5. The size of the dose is typically illustrated in a window 26 provided in or on the body 6 of the injection device 1. Prior to inject a dose of the medicament 24 the distal end of the container part 7 or cartridge holder has to be connected with a needle assembly 12. For this, the distal end of the cartridge holder 7 comprises a connector 11, e.g. in form of a threaded interface to engage with a complementary shaped threaded counter interface of the needle assembly 12.

The needle assembly 12 is detachably or releasably fixable to the container part 7. It comprises a double-tipped injection needle 13. A proximal end of the injection needle (not shown) is configured to enter into a through opening at the distal end face of the connector 11 or container part 7 so as to pierce or to penetrate the seal 23 of the medicament container 21. The distal end of the injection needle 13 is typically covered by a detachable inner needle cap 14. The entirety of the needle assembly 12 may be covered by a detachable outer needle cap 15.

The container part 7 and hence a portion of the housing 10 is to be received in a protective cap 16, which is detachably connectable to the cartridge holder 7 or body 6.

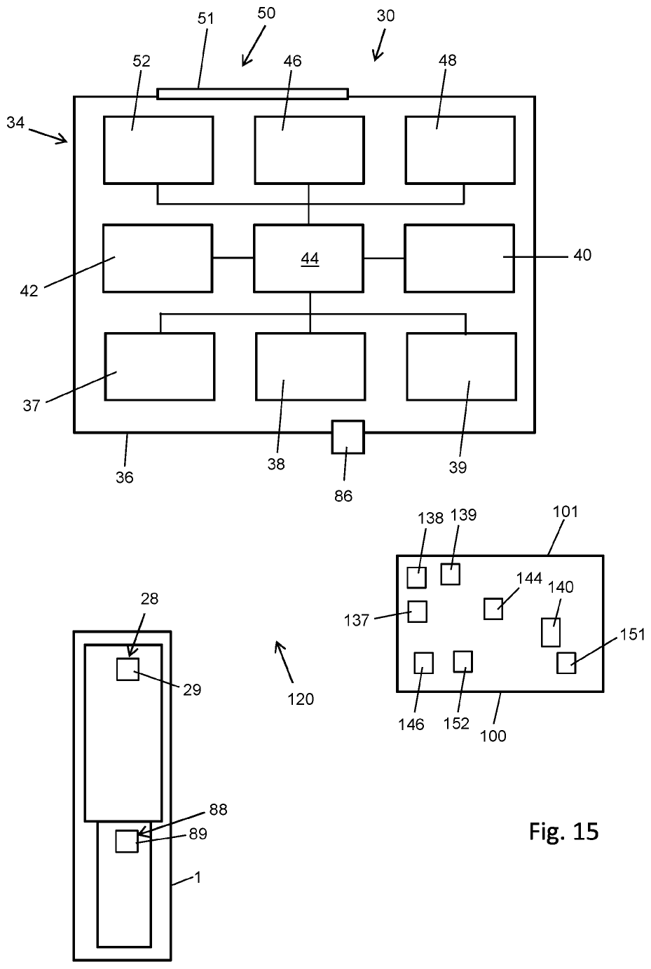

In FIGS. 2 and 4 there is shown an example of an add-on device 30 configured for fastening to the proximal end of the injection device 1. The add-on device 30 comprises a sensor 48 or sensor assembly, which is operable to detect, to recognize, to characterize and/or to measure an operation of the injection device 1. Typically, the sensor 48 of the add-on device 30 and as indicated in the block diagram of FIG. 15 is capable or operable to quantitatively determine or to measure at least one of a position and a movement of a movable component 80, 81, 82, 83 of the drive mechanism 20 of the injection device 1. The movable component 80, 81, 82, 83 is movable relative to at least one of the body 6 and the medicament container 21 during or for setting and/or injecting of a dose of the medicament 24.

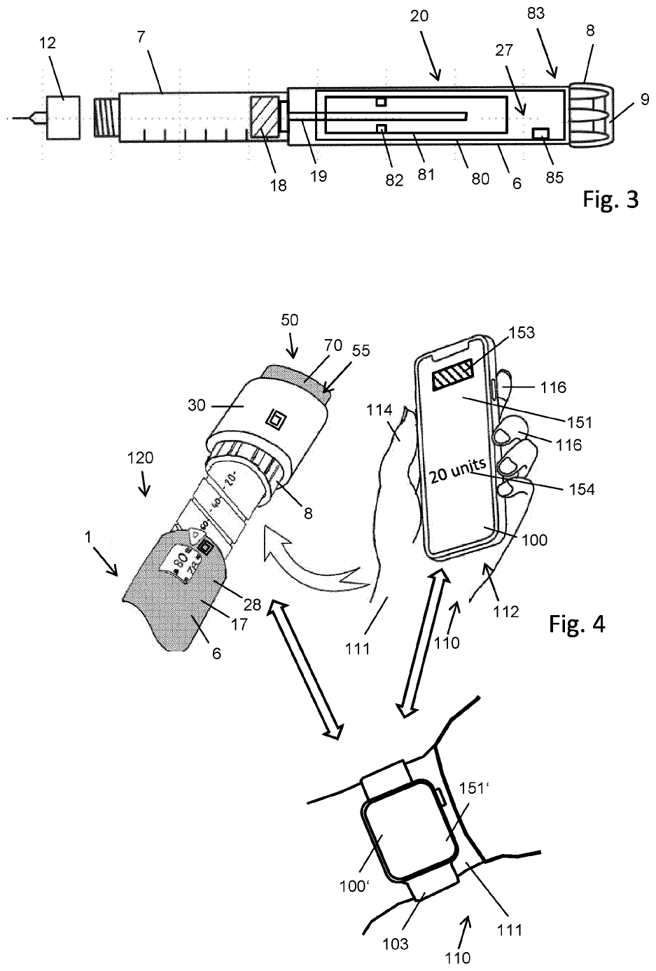

This movement or position is quantitatively measurable by the sensor 48. Typically, the sensor 48 is implemented as an electronic sensor. It is capable to detect or to measure a degree of longitudinal translation and/or rotation of the at least one movable component 80, 81, 82, 83 relative to the housing 10 or relative to the device body 60 when the add-on device 30 is attached to the injection device 1. Examples of movable components 80 are schematically illustrated in the cross-section of the injection device 1 according to FIG. 3 The sensor 48 of the add-on device 30 is capable or operable to generate a sensor signal or a sequence of sensor signals, which due to the coupling or connection to the module processor 44 can be processed by the module processor 44 in order to derive or to calculate a size of a dose currently set or dispensed.

With some examples the movable component 80 is a dose setting member or a dose setting sleeve. With further examples the movable component 81 is implemented as a drive member or drive sleeve 81 of the drive mechanism 20. With further examples the movable component is implemented as a volume indicator 82, e.g. as a single dose indicating member, whose position or configuration relative to the body 6 is directly indicative of the size of a dose currently set.

With further examples the movable component 82, hence the volume indicator is a last dose indicating member, the position or configuration of which relative to the body 6 is indicative of a remaining amount of medicament left in the cartridge or medicament container 21.

With some examples the movable component 83 is or comprises an encoding, such as a digital encoding, which is movable relative to the sensor 48 such that the sensor is capable to count a number of discrete code portions of the encoding so as to gather respective quantitative movement information of the movable component 80. Generally, there may be provided different types of spatial encodings, such as an optical or visual encoding, an electrical or electrostatic encoding a magnetic encoding or mechanical encoding. Depending on the type of an encoding provided on or inside the movable component 80, 81, 82, 83 the sensor 48 is correspondingly configured. Hence, the sensor 48 may comprise at least one of an optical sensor, an electrical or electrostatic sensor and a magnetic sensor or mechanical sensor, such as a micromechanical switch, each of which being operable to quantitatively measure a relative position or relative movement of the movable component 80, 81, 82, 83.

With some examples the movable component 80, 81, 82, 83 is subject to a longitudinal and/or rotational movement relative to the body 6, relative to the medicament container 21 and/or relative to the device body 60 during at least one of setting of the dose and dispensing of the dose. Typically, the encoding as provided on or with the movable component 80, 81, 82, 83 is encoded along the direction of movement of the movable component relative to the sensor 48 so as to enable a respective quantitative measurement of the respective dose setting or dose dispensing movement.

As further indicated in FIG. 3 the drive mechanism 20 may be optionally equipped with a locking mechanism 85, which is operable by a locking controller of the add-on device 30 when the add-on device 30 is correctly assembled to the injection device 1. The locking mechanism 85 is operable to block or to lock the drive mechanism 20 so as to prevent unauthorized or unintended setting of a dose and/or dispensing or injecting of the dose. The locking mechanism 85 is controllable by the locking controller 86 of the add-on device 30. The locking mechanism 85 may be implemented electromechanically and may be electrically or electronically controlled by the locking controller 86 of the add-on device 30. This way, the add-on device 30 may block an unauthorized or unintended use of the injection device 1.

The add-on devices 30 as illustrated in the various FIGS. 2-15 is detachably connectable to the dose dial 8. It comprises a device body 60 with a tubular-shaped sidewall 61. Towards the distal end the sidewall 61 confines a receptacle 63, which is sized to receive the dose dial 8 and the trigger 9 of the injection device 1. For this, the inside of the sidewall 60 may comprise one or numerous fastening ribs 64, which are configured to provide a slip free fastening of the add-on device 30 to the dose dial 8.

The device body 60 comprises the receptacle 63 at a distal end section of the add-on device 30. The receptacle 63 is open towards the distal direction 2. It is sized and configured to fit onto the proximal end of the injection device 1. An inside surface of the sidewall 61 confining the receptacle 63 comprises numerous fastening ribs 64, e.g. of an elastic or elastically deformable material, such as an elastomeric material.

The fastening ribs 64 provide a friction fit with the dose dial 8 when the device body 60 is assembled to the dose dial 8 of the injection device 1. The receptacle 63 may be confined in longitudinal direction by a flange portion 62 protruding radially inwardly from the tubular-shaped sidewall 61 of the receptacle 63. The flange portion 62 is located proximally offset from the insert opening of the receptacle 63. The flange portion 62 may be of annular shape and may be configured to axially abut on a stepped down section at the proximal end of the dose dial 8.

Hence, the trigger 9, which comprises a reduced diameter compared to the dose dial 8 may protrude in proximal direction 3 through the flange portion 62 while the flange portion 62 may rest or abut against the proximal end face of the dose dial 8 when the add-on device 30 is suitably fastened to the dose dial 8.

The add-on device 30 further comprises a movable part 70 protruding in proximal direction from the device body 60. The movable part 70 comprises or forms an auxiliary trigger or trigger button, which is configured to mechanically engage with the trigger 9 of the injection device 1 when the add-on device 30 is correctly assembled to the injection device 1.

Typically, the add-on device 30, in particular, the device body 60 is frictionally engageable with the dose dial 8. In this way, a user may apply a dose setting torque onto the dose dial via the device body 60. Instead of rotating the dose dial 8 for setting of a dose the user may simply rotate the device body 60 relative to the body 6 of the injection device 1. This way, the dial extension 27 may become subject to a dose incrementing dialing or rotating motion. A respective size of a dose currently set will then be displayed in the window 26 of the body 6 of the injection device 1 as illustrated in FIG. 2.

For dispensing of a dose the user has to depress the movable part 70, which may then be subject to a distally directed motion relative to the device body 60. The movable part 70, which may be in direct or indirect mechanical engagement with the trigger 9, may then apply a respective dispensing force onto the trigger 9 thereby initiating a dose dispensing action of the injection device 1.

The add-on device 30 as schematically illustrated in FIG. 15 comprises an electronic module 34. The electronic module 34 comprises a printed circuit board 36. The electronic module 34 comprises a module processor 44, an electronic and hence digital module memory 40 and a clock 42. Furthermore, the electronic module 34 comprises a power source 46 and the sensor 48. The electronic module 34 also comprises a signal generator 52 coupled to the processor 44 and/or coupled to the power source 46. The electronic module 34 further comprises at least one of a reader 37 and a wireless transceiver 38, 39. The add-on device 30 may also comprise a user perceptible device identification 50.

With some examples the device identification 50 comprises or includes the signal generator 52 coupled to the module processor 44 and operable or reconfigurable by the module processor 44. With some examples the device identification 50 may be indicative of the injection device 1 to which the add-on device 30 is actually connected or coupled to.

With some examples the signal generator 52 is operable to generate at least one of a visual, an acoustic and a haptic signal or device identification and is hence operable to produce or to generate a visual identifier, an acoustic identifier or a haptic identifier or a respective alert signal. With some examples the add-on device 30 comprises numerous signal generators 52 of equal or different type, e.g. optical, acoustic or haptic type.

With the example of FIG. 2 the signal generator 52 may comprise a speaker operable to generate an acoustic signal, wherein the acoustic signal may represent or constitute an alert signal e.g. in form of an acoustic code or sequence being characteristic for a specific configuration or setting of the injection device 1 or add-on device 30.

FIG. 4 is illustrative of an injection system 120 comprising at least the add-on device 30 and the injection device 1. Optionally, the injection system 120 comprises one or several external electronic devices 100, 101′. The external electronic device 100 is implemented as a smart phone comprising a housing 101 and being equipped with a device display 151 to visually illustrate various types of information, such as various illustrations 153 or notifications 154 to a user. The display 151 may be implemented as a touch sensitive display. The device display 151 may be operable to provide or to emulate a device signal generator 152. The device display 151 is operable to provide at least one of an illustration 153 and a notification 154 to a user.

The further optional external electronic device 100′ is implemented as a smartwatch comprising a respective housing and a display 151′ and further comprising a wristband 103 for fixing the external electronic device 100′ to the hand 110 or wrist 111 of a user. Like the external electronic device 100 also the external electronic device 100′ is operable to provide at least one of an illustration 153 and a notification 154 to the user. Generally, the external electronic device 100, 100′ may be equipped with a separate device signal generator 152, such as a speaker or a vibration module by way of which a user perceivable signal can be generated so as to provide an alert signal to a user of the respective device

The electronic device 100 is typically implemented as a handheld electronic device. It can be held in a hand 110 of a user or may be worn on the wrist 111 of a user. With the example as illustrated in FIG. 4 the electronic device 100 is held in a palm 112 of the user. The device display 151 is implemented as a touch sensitive display and can be operated by a thumb 114 and/or by various fingers 116 of the user's hand 110.

The electronic module 34 of the add-on device 30 as schematically illustrated in FIG. 15 comprises at least one of a reader 37 and a transceiver 38, 39 operable for wireless communication with at least one of a machine-readable identification 28, 88 of the injection device 1 or with a complementary or correspondingly implemented device reader 137 and/or device transceiver 138, 139 of the external electronic device 100, 100′.

With typical examples the transceivers 38, 138 are implemented as radiofrequency transceivers of near field type. The transceivers 38, 138 may be implemented as wireless short range transceivers or near field transceivers. They may be implemented as a so-called NFC transceivers allowing for wireless communication within a limited spatial range of a few centimeters or decimeters. The transceivers 38, 138 may be implemented as active and/or passive NFC tags or readers. Typically, the transceiver 38 is implemented as a passive NFC tag and the transceiver 138 is implemented as an active NFC tag or NFC reader.

The transceivers 39, 139 may be implemented as wireless local range transceivers, such as RFID, Bluetooth, Bluetooth low energy (BLE) or UWB transceivers. Also here, the transceiver 39 may be implemented as a passive communication tag whereas the transceiver 139 may be implemented as an active transceiver, hence as a reader. Typically, the transmission range of the transceivers 39, 139 is larger than the transmission range of the transceivers 38, 138. The transmission range of the transceivers 39, 139 may be in a range of several meters or decameters.

The optional reader 37, 137 may be implemented as an optical reader or optical information gathering unit, e.g. comprising an imaging system so as to capture or to record a visual or optical machine-readable identification 28, 88 as provided on or inside the injection device 1.

The external electronic device 100, 100′ typically comprises a device processor 144 coupled to an electronic device memory 140. The electronic device 100 further comprises a device power source 146. Also, the electronic device 100 comprises a device display 151, typically implemented as a touch sensitive display. The device display 151 may be operable to provide at least one of an illustration 153 and a notification 154 to a user.

The device transceiver 138 is operable to communicate with the transceiver 38 of the add-on device. The transceiver 139 is configured to communicate with the transceiver 39 of the add-on device 30. The reader 137 may be operable to read a visual identifier as provided on the injection device 1. With some examples the device reader 137 comprises an imaging system. It may comprise a camera objective and a spatially resolving detector in order to read or to capture a visual code, e.g. provided on an outside surface of the injection device 1 or as provided on an outside surface of the medicament container 21.

The external device 100 is configured to set up a communication link, e.g. a wireless communication link with the add-on device 30. For this, the local range transceiver 39 of the add-on device 30 may communicate wirelessly with the local range device transceiver 139 of the add-on device 100. Once a communication link has been established between the electronic device 100 and the add-on device 30 the electronic device 100 may be operable to visibly illustrate information or data of the injection device or medicament container 21 on the device display 151

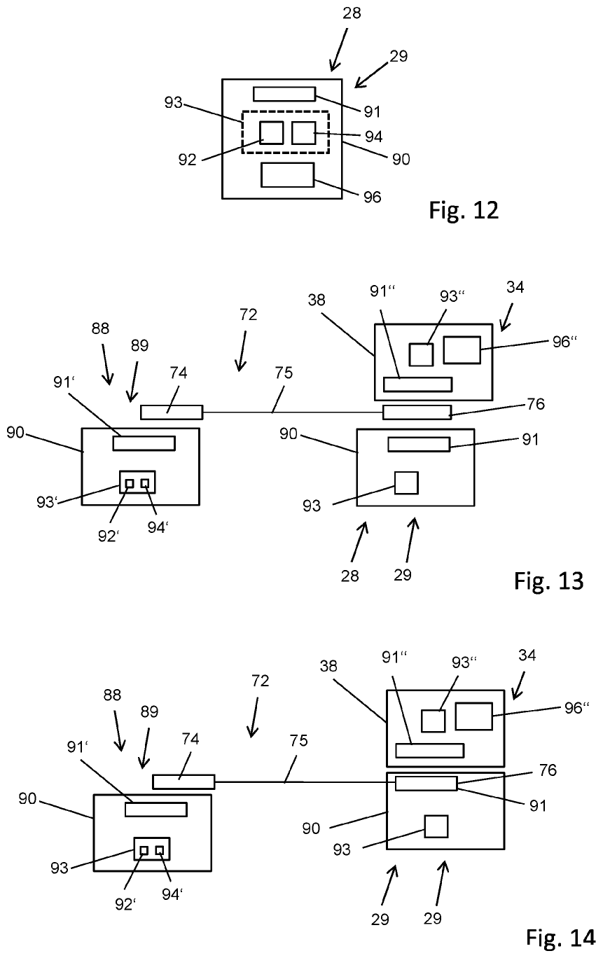

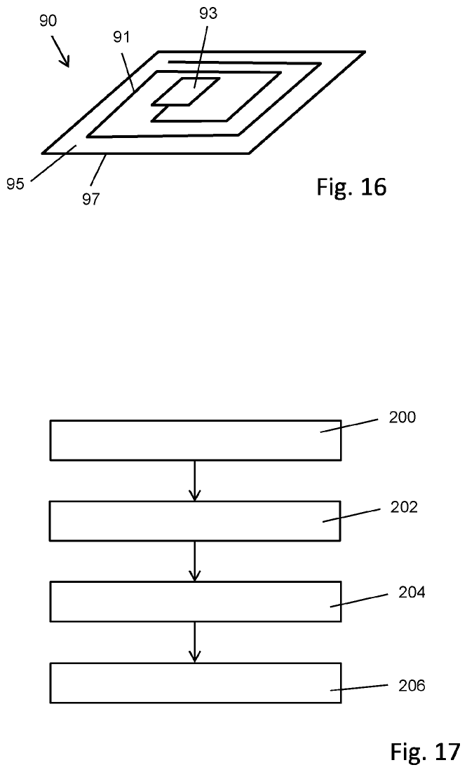

The injection device 1 is provided with a machine-readable identification 28, 88 as schematically illustrated in FIG. 15. The machine-readable identification 28 comprises an electronic identifier 29. The machine-readable identification 88 comprises an electronic identifier 89. The electronic identifier 28 may comprise a passive wireless communication tag as schematically illustrated in FIG. 12 or 16.

As illustrated in FIGS. 12 and 16 the electronic identifier 29 may comprise an electronic circuit 90 featuring a wireless communication antenna 91 and an integrated circuit 93. The integrated circuit 93 comprises an electronic and hence digital memory 92 to and a processor 94 connected to the memory 92. Of course, the integrated circuit 93 and hence the processor 94 is connected to the antenna 91. The electronic identifier 29 may optionally comprise a power source 96 in order to provide electrical power or energy for the processor 94. The electronic circuit 90 may be provided on a planar substrate 95. The substrate 95 may be pliable or foldable. It may comprise a flexible structure or foil that allows for an easy mounting, fastening e.g. adhesive fastening, of the substrate 94 and hence of the entire electronic identifier 29 to at least one of the tubular shaped body 6, the container part 7 or the medicament container 21.

The electronic circuit 90 may be a printed electronic circuit, which is printed on the substrate 95. Here, the substrate 95 may comprise or constitute an e.g. flexible printed circuit board. The substrate 95 may be further provided with an adhesive layer 97 that allows for an easy and straightforward adhesive attachment of the machine-readable identification 28, 88 to a predefined portion on or inside the injection device 1. The electronic identifier 89 may comprise an identical or similar structure as the electronic identifier 29 as shown in FIGS. 12 and 16.

With some examples the machine-readable identification 28, 88 is implemented in a label 17 located on one of the body 6, the container part 7 or the medicament container 21. The label 17 comprises or contains the machine-readable identification 28, 88. With some examples the label 17 comprises a passive radiofrequency tag. With further examples the label 17 comprises machine-readable identifications 28, 88 implemented as a visual or optical code that can be captured by a reader 37 or device reader 137, which is implemented as or comprises an imaging system.

At least one of the reader 37, the device reader 137 or at least one of the transceivers 38, 39 or device transceivers 138, 139 is or are operable to read the machine-readable identification 28, 88 thereby obtaining at least one of a device information and a container information e.g. stored in the memory 92 of the electronic circuit 90.

Typically, the machine-readable identification 28 is provided or located on or inside the body 6 of the housing 10 of the injection device 1. The further machine-readable identification 88 is provided on or inside the container part 7. It may be provided on or inside the medicament container 21. With some examples the machine-readable identification 28 may be provided in or on the piston 18 of the medicament container 21.

The machine-readable identification 28, 88 may be implemented as a passive RF communication tag, such as a NFC tag. The information, e.g. the device information and/or the container information stored in at least one of the machine-readable identification 28, 88 is readable by the electronic module 34 and hence by any one of the transceivers 38, 39 of the add-on device 30 and/or by any of the transceivers 138, 139 of the external electronic device 100, 100′.

With some examples and especially when the injection device 1 is implemented as a disposable injection device, which does not support replacement of the medicament container 21 it is sufficient when the injection device 1 comprises only one machine-readable identification 28. It may be of particular advantage when the machine-readable identification 28 is provided at or near a proximal end 68 of the body 6. It may be provided on or inside the dose dial 8 or the trigger 9 so as to be within the transmission range of the transceiver 38, 39 when the add-on device 30 is attached to the proximal end of the injection device 1.

With some examples the distal end 67 of the body 6 is detachably connectable to a proximal end of the container part 7. Then, the injection device 1 is implemented as a reusable device that allows a replacement of the container part 7 and/or a replacement of the medicament container 21 inside the container part 7. With reusable injection devices 1 it may be of particular benefit when both, the body 6 and the container part 7 are provided with a separate machine-readable identification 28, 88. As illustrated in FIG. 15, the container part 7 is provided with the machine-readable identification 88 whereas the body 6 is provided with the machine-readable identification 28.

With some examples the transceiver 38 may be located inside the receptacle 63 of the device body 60 so as to minimize a spatial distance to the machine-readable identification 28 as provided at or near the proximal end 68 of the body 6. By making use of near field communication technology for the machine-readable a notification 28, 88 and the respective transceivers 38, 138 it can be ensured, that the machine-readable identification and the device information and/or container information stored therein can only be acquired or obtained when the add-on device 30 and/or the external electronic device 100, 100′ is within the near field transmission range.

As further illustrated in FIG. 12, the receptacle 63 of the device body comprises an alignment feature 65 in form of a longitudinal recess or groove on the inside of the sidewall of the receptacle 63. Correspondingly, the proximal end of the dose dial 8 comprises a complementary shaped counter alignment feature 66, e.g. in form of a protrusion sized to fit into the recess or groove of the alignment feature 65. In this way, there can be provided a rotation inhibiting fastening for the add-on device 30 when attached to the housing 10 or dose dial 8 of the injection device 1.

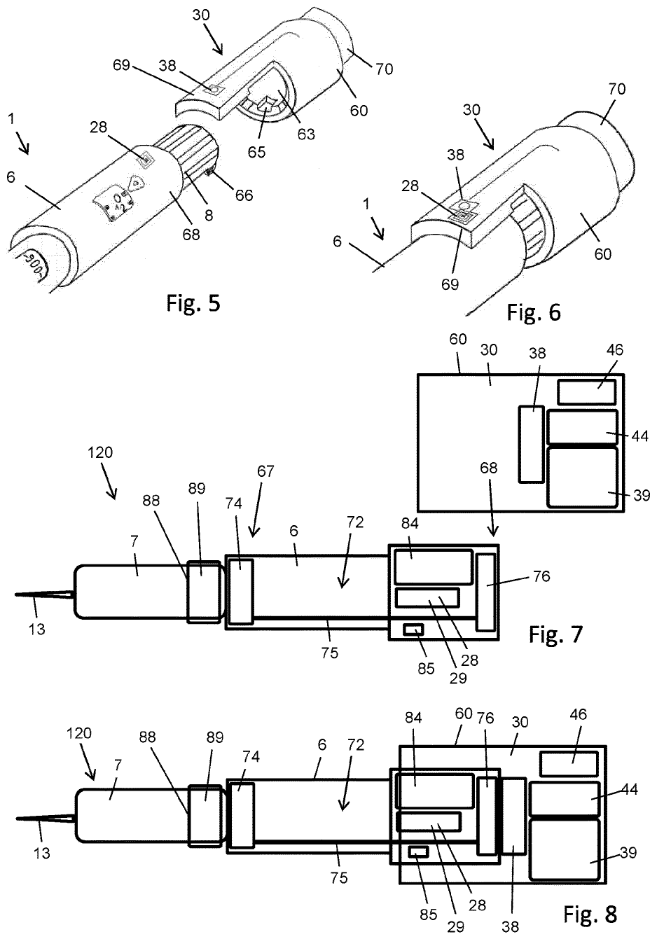

When the alignment feature 65 is in mechanical engagement with the counter alignment feature 66 as shown in FIG. 5 there is provided an anti-rotation lock for the mutual fastening of the add-on device 30 to the injection device 1. In this way there can be guaranteed, that the identification 28 and hence the electronic identifier 29 always correctly aligns with the associated wireless transceiver 38 of the add-on device 30. There is hence provided a unique and well-defined mounting position for mounting the add-on device 30 to the injection device 1. When reaching the mounting position or mounting configuration as predefined by alignment feature 65 engaging the counter alignment feature 66 it is also guaranteed, that the machine-readable identification 28 and the transceiver 38 are at a minimum distance to each other. This allows for a somewhat undisturbed signal transmission between the identification 28 or electronic identifier 29 and the transceiver 38.

Generally, the external electronic device 100 may permanently or regularly synchronize with the add-on device e.g. by local range communication interfaces as provided by the transceivers 39, 139. Insofar, any information gathered or read by the add-on device 30 can be instantly transmitted to and further processed, e.g. visualized, by the external electronic device 100, 100′.

In FIGS. 7-14 there are illustrated numerous examples of an injection the device 1 equipped with a first antenna 74 and a second antenna 76. The first antenna 74 is electrically connected to the second antenna 76. The first and the second antennas 74, 76 are separated in longitudinal direction. The first antenna 74 is provided at or near a distal end 67 of the body 6. The second antenna 76 is provided at or near a proximal end 68 of the body 6 or of the drive mechanism 20.

With some examples the second antenna 76 may be provided on or inside the trigger 9 or dose dial 8 of the drive mechanism 20. The first antenna 74 and the second antenna 76 are connected by an electrical transmission line 75. In case that the second antenna 76 should be movable relative to the first antenna 74, the transmission line 75 may comprise at least one or several sliding contacts for maintaining the electrical connection between the first antenna 74 and the second antenna 76. With some examples the first antenna 74 interconnected to the second antenna 76 via the transmission line 75 may constitute a bridge circuit 172 effectively providing a kind of a range extender for the wireless transmission of the second antenna 76.

The first antenna 74 is operable to wirelessly communicate with the electronic container identifier 89 and hence with a machine-readable identification 88 as provided on one of the container part 7 and the medicament container 21. The electronic container identifier 89 may be provided at or near the proximal end of the container part 7 or at or near a proximal end of the medicament container 21. When the container part 7 is correctly attached and connected to the body 6, the electronic container identifier 89 may radially overlap with the first antenna 74. The electronic container identifier 89 and the first antenna 74 may be arranged concentrically.

The second antenna 76 is configured for wireless communication with at least one of the transceiver 38, 39 of the electronic module 34 of the add-on device 30. Signal transmission between the transceiver 38, 39 with the second antenna 76 may be enabled as the add-on device 30 is attached to the proximal end 68 of the body 6 or injection device 1 as illustrated in FIG. 8. Then, a distance between the device transceiver 38 and the second antenna 76 may be smaller than the transmission range of the device transceiver 38.

With some examples the device transceiver 38 is implemented as a near field communication interface operable to wirelessly communicate with a complementary near field communication antenna 76.

When assembled or attached to the injection device 1, the distance between the first antenna 74 and the transceiver 38 may be larger than the transmission range of the transceiver 38.

Here, and in order to provide signal transmission between the first antenna 74 and the transceiver 38 the first antenna 74 is connected to the second antenna 76 via the transmission line 74 extending between the first and the second antennas 74, 76.

With the example as shown in FIGS. 7 and 8 the injection device 1 is further equipped with an electronic device identifier 28. The electronic device identifier 28 comprises an electronic circuit 90 with an antenna 91 and an integrated circuit 93. Here, the antenna 91 is or represents an identifier antenna of the electronic device identifier 29. The transceiver 38 may be operable to communicate with both, the second antenna 76 as well as with the antenna 91 of the machine-readable identification 28 and hence the electronic device identifier 29 of the injection device 1.

The electronic module 34 is operable to read or to obtain container information as provided by a memory 92′ of the electronic container identifier 89. Likewise, the electronic module 34 is operable to read and/or to obtain device related information from the memory 92 of the electronic device identifier 29. The electronic module 34 is operable to evaluate the device information and the container information and to compare the respective information in order to check or to assert if the medicament container 21 or the container part 7 matches with the drive mechanism 20 and/or with the body 6.

In case that an incorrect matching is to be detected by comparing the device information obtained from the electronic device identifier 29 and the container information as obtained from the electronic container identifier 89 the electronic module 34 is operable to activate at least one of an alert and a locking mechanism 85 of the drive mechanism 20.

With the example as illustrated in FIGS. 7 and 8 the second antenna 76 is exclusively connected to the first antenna 74 via the transmission line 75. Hence, the second antenna 76 is exclusively used to provide the reading of the electronic container identifier 89 when the add-on device 1 is attached to the proximal end of the injection device 1 and when the container part 7 is attached to the distal end of the body 6.

The transmission line 75 and/or the antennas 74, 76 are further operable to transmit electric power from the transceiver 38 to the first antenna 74, which electric power is sufficient and operable to induce a wireless response from the electronic container identifier 89. Here, the electronic container identifier may be implemented as a passive wireless communication tag. In this way, the electronic device identifier 89 does not require an own power source.

An example of the wireless electronic communication between the external electronic device 30, the electronic device identifier 29 and the electronic container identifier 89 is further illustrated in FIG. 13. Both, the electronic device identifier 29 and the electronic container identifier 89 comprise an electronic circuit 90. The electronic circuit 90 of the electronic container identifier 89 comprises an integrated circuit 93′ and an antenna 91′. The integrated circuit 93′ comprises a memory 92′ and a processor 94′. Likewise, the electronic device identifier 29 comprises an electronic circuit 90 with an antenna 91 and an integrated circuit 93. The integrated circuit 93 itself and/or its hardware implementation may resemble the respective structure or hardware implementation of the integrated circuit 93′; and vice versa. Insofar, also the integrated circuit 93 comprises a processor 94 and a memory 92 as described in greater detail with regard to FIG. 12 with the example of the electronic device identifier 29.

The first antenna 74 is connected to the second antenna 76 via the transmission line 75. The antenna 91′ of the container identifier 89 is in the transmission range of the first antenna 74. It may be outside the transmission range of the second antenna 76. It may be also outside the transmission range of the antenna 91″ of the transceiver 38 of the add-on device 30 when the add-on device 30 is attached to the injection device 1.

The antenna 91 of the electronic device identifier 29 is within the transmission range of the second antenna 76. It may be outside of the wireless transmission range of the antenna 91′ of the electronic container identification 89. The antenna 91 may be operable to directly wirelessly communicate with the antenna 91″ of the transceiver 38 of the add-on device 30. With some examples the antenna 91 may be operable to wirelessly communicate with the second antenna 76. Here, the transceiver 38 is provided with a wireless signal anti-collision mechanism so as to distinguish between signals received from the antenna 91′ and signals received from the antenna 91. Insofar, the transceiver 38 and/or the module processor 44 is or are operable to provide a demultiplexing of wireless data transmission with the antennas 91, 91′. With other examples the antennas 91, 91′ may be operated with different communication protocols that inherently provide a respective signal separation.

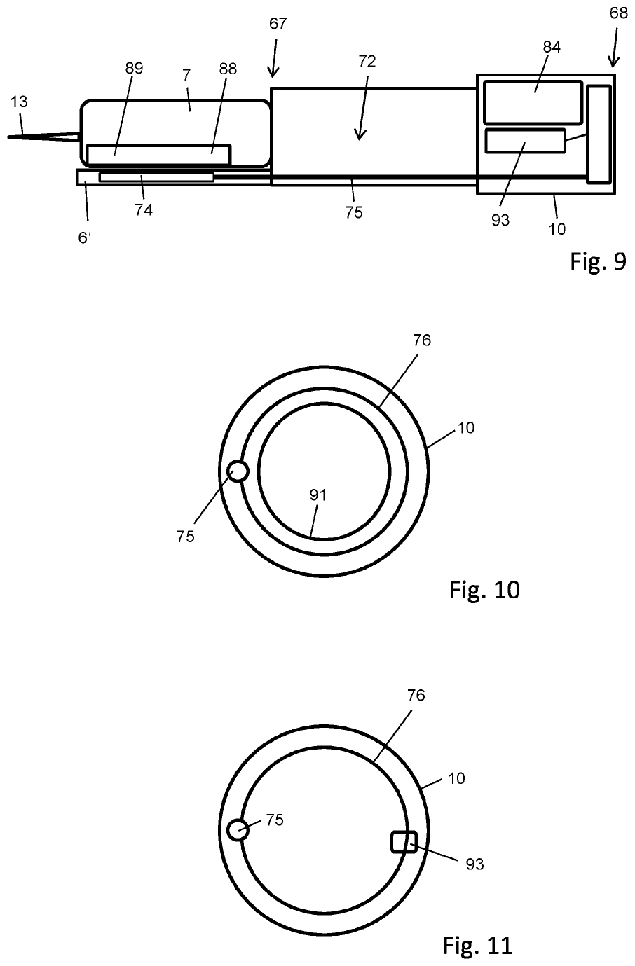

With the further example as illustrated in FIG. 14 which also reflects in the example of FIG. 9, the second antenna 76 may be integrated into the electronic circuit 90 of the electronic device identifier 29. Here, the electronic device identifier 29 may be void of an own antenna but is operable to exclusively make use of the second antenna 76 to communicate with the transceiver 38. Here, the transceiver 38 also has an anti-collision and/or a respective multiplexing mechanism so as to distinguish from signals obtained from the electronic container identifier 89 and signals obtained from the electronic device identifier 29.

The transceiver 38 of the electronic module 34 of the add-on device 30 may be implemented as a RFID reader, NFC reader and/or UWB reader. It comprises an antenna 91″, an integrated circuit 93″ and optionally also known power source 96″. Generally, the integrated circuit 93″ may comprise the module processor 44 and the power source 96″ may be provided by the power source 46 of the electronic module 34.

With some examples the injection device 1 is equipped with a dose detection arrangement 84. The dose detection arrangement 84 may comprise a movable component 80, 81, 82, 83 whose position or movement relative to at least one of the housing 10, the medicament container 21 and/or the device body 60 can be detected and/or quantitatively measured, e.g. by the sensor 48 of the electronic module 34 of the add-on device 30.

In FIG. 10 there illustrated a concentric arrangement between the antenna 91 of the electronic device identifier 29 and the second antenna 76 at or near the proximal end of the body 6. Here, the second antenna 76 is connected or connectable to the first antenna 74 via the longitudinally extending transmission line 75. The concentric arrangement of the antennas 91, 76 is of particular benefit for an effective signal transmission.

In FIG. 11 there is provided a schematic cross-section through the device configuration according to FIGS. 9 and 14. There, the second antenna 76 is connected to the first antenna 74 by the transmission line 75 and the second antenna 76 is further directly connected to the integrated circuit 93 of the electronic device identifier 29. It is to be noted that the illustration of FIG. 11 is only of schematic nature. The integrated circuit 93 may be located inside the circumference of the antenna 76, the latter of which may be implemented as a loop antenna 76. Accordingly, the integrated circuit 93 may be located coaxially inside the surrounding antenna 76.

With the further example as illustrated in FIG. 9 the body 6 of the injection device 1 is provided with a longitudinal body extension 6′ protruding distally from the distal end 67 of the tubular portion of the body 6. The longitudinal body extension 6′ is provided with the first antenna 74, which is connected to the second antenna 76 via the transmission line 75. Arranging the first antenna 74 distally from the tubular portion of the body 6 may provide a benefit to reduce the geometrical distance between the first antenna 74 and the electronic container identifier 89.

Here, the electronic container identifier 89 can be provided on a sidewall of the container part 7 and/or on the sidewall of the barrel 22 of the medicament container 21. The electronic container identifier 89 may be provided by or on a label 17 that is e.g. adhered to and inside or outside surface of the container part 7 or which is adhered on an outside surface of the barrel 22.

Arranging of the electronic container identification 89 on a sidewall of at least one of the medicament container 21 and the container part 7 allows to use a comparatively large antenna 91′, e.g. in form of a rectangular label antenna, which improves and facilitates respective readout of the electronic container identifier 89.

FIG. 17 is illustrative of a flowchart of a method of monitoring operation of the injection device 1. Here, in a first step 200, the add-on device 30 is attached to the injection device 1. thereafter and in a second step 202 the container information as provided by the electronic container identifier is read or obtained by the electronic module 34 of the add-on device 30.

Reading of the container information is provided through wireless signal transmission between the transceiver 38 and the second antenna 77, which is connected to the first antenna 74 through the transmission line 75 of the injection device 1. The first antenna 74 wirelessly communicate with the electronic container identifier 89, which is provided on or inside one of the medicament container 21 and the container part 7 of the injection device 1.

In a further step 204 device information from the electronic body identifier 29 is obtained or read by the electronic module 34. Also here, the transceiver 38 is operable to read respective device information from the memory 92 of the electronic body identifier 29.

After having obtained device information from the electronic body identifier 29 and after having obtained container information from the electronic container identifier 89 step 206 the respective device information and container information is evaluated. Here, information or data about the medicament container 21 or medicament 24 can be compared with respective information about the body 6 and/or the drive mechanism 20.

As a result of the comparison or evaluation of the device information and the container information the electronic module 34 may approve or disapprove use of the injection device 1, e.g. by unlocking the locking mechanism 85 through the locking controller 86. With further examples and in response to an evaluation of device information and container information the electronic module 34 may be operable to generate an alert or any other indication to a user thus informing the user about the status of the injection device 1 and/or the add-on device 30. In addition, data obtained from any of the electronic container identifier 89 and the electronic device identifier 28 can be synchronized with an external electronic device 100, 100′ in order to provide respective information to a user.

### REFERENCE NUMBERS

1 injection device
2 distal direction
3 proximal direction
4 dose incrementing direction
5 dose decrementing direction
6 body
6′ body extension
7 container part
8 dose dial
9 trigger
10 housing
11 connector
12 needle assembly
13 injection needle
14 inner needle cap
15 outer needle cap
16 protective cap
17 label
18 piston
19 piston rod
20 drive mechanism
21 medicament container
22 barrel
23 seal
24 medicament
25 outlet
26 window
27 dial extension
28 identification
29 electronic device identifier
30 add-on device
34 electronic module
36 integrated circuit
37 reader
38 transceiver
39 transceiver
40 module memory
42 clock
44 module processor
46 power source
48 sensor
50 indicator
51 display
52 signal generator
53 display section
54 display section
60 device body
61 sidewall
62 flange portion
63 receptacle
64 fastening rib
65 alignment feature
66 counter alignment feature
67 distal end
68 proximal end
69 extension
70 movable part
72 bridge circuit
74 antenna
75 transmission line
76 antenna
80 dose setting element
81 drive member
82 volume indicator
83 encoding
84 dose detection arrangement
85 locking mechanism
88 identification
89 electronic container identifier
90 electronic circuit
91 identifier antenna
92 memory
93 integrated circuit
94 processor
95 substrate
96 power source
97 adhesive layer
100 electronic device
101 housing
103 wristband
110 hand
111 wrist
112 palm
114 thumb
116 finger
120 injection system
137 device reader
138 device transceiver
139 device transceiver
140 device memory
144 device processor
146 device power source
151 device display
152 device signal generator
153 illustration
154 notification

1-15. (canceled)

16. An injection device for injecting of a dose of a medicament, the injection device comprising:
a housing comprising a body detachably connectable to a container part, wherein the container part is configured to accommodate a medicament container,
a drive mechanism arranged in the body and operable to expel the dose of the medicament from the medicament container,
a first antenna on or in a first portion of the body, which is configured for wireless signal transmission with an electronic container identifier provided on or in the container part,
a second antenna on or in a second portion of the body electrically connected to the first antenna and configured for wireless signal transmission with a transceiver of an electronic module of an add-on device connectable to the injection device,
an electronic device identifier on or inside the body and configured for wireless signal transmission with the transceiver.

17. The injection device according to claim 16, wherein the electronic device identifier comprises an integrated device circuit with a device memory configured to store at least one of a device information and a use-related data.

18. The injection device according to claim 17, wherein for wireless signal transmission between the integrated device circuit and the transceiver:
(i) the integrated device circuit is electrically connected to the second antenna, or
(ii) the integrated device circuit is electrically connected to an identifier antenna of the electronic device identifier, wherein the identifier antenna is configured for wireless signal transmission with the transceiver.

19. The injection device according to claim 16, wherein the body comprises a distal end connectable to the container part and wherein the body comprises a proximal end opposite the distal end, wherein the first antenna is arranged at or near the distal end and wherein the second antenna is arranged at or near the proximal end.

20. The injection device according to claim 16, further comprising the container part and wherein the electronic container identifier is arranged on or in one of the container part and the medicament container accommodated by the container part.

21. The injection device according to claim 20, wherein the electronic container identifier comprises a container antenna and an integrated container circuit connected to the container antenna, wherein the integrated container circuit comprises a container memory configured to store at least one of a container information and a use-related data.

22. The injection device according to claim 16, wherein the container memory is readable by the transceiver of the electronic module of the add-on device when attached to the injection device, through wireless signal transmission between the first antenna and the container antenna and through wireless signal transmission between the second antenna and the transceiver.

23. The injection device according to claim 16, wherein at least one of the electronic container identifier and the electronic device identifier comprises a passive RFID tag.

24. An add-on device for attaching to an injection device according to claim 16, the add-on device comprises:
a device body fastenable to a portion of the injection device,
an electronic module comprising a module processor and a transceiver, wherein the electronic module is operable:
to read data from the electronic container identifier via the first antenna and the second antenna, and
to read data from the electronic device identifier wirelessly.

25. The add-on device according to claim 24, wherein the transceiver is configured for wireless signal transmission with the second antenna when the device body is fastened to the injection device.

26. The add-on device according to claim 24, wherein the transceiver is configured for wireless signal transmission with an identifier antenna of the electronic device identifier when the device body is fastened to the injection device.

27. The add-on device according to claim 24, wherein the electronic module is operable to separate and/or to distinguish between signals received from the electronic container identifier and the electronic device identifier.

28. The add-on device according to claim 24, wherein the electronic module is operable to induce and to transmit electric power to the first antenna via the second antenna and a transmission line connecting the first antenna and the second antenna, wherein the electric power is effective to induce signal transmission between the first antenna and the electronic container identifier.

29. The add-on device according to claim 24, further comprising a sensor operable to quantitatively determine at least one of a position and a movement of a movable component relative to at least one of the housing, the medicament container and the device body and operable to generate a respective sensor signal, wherein the module processor is operable to determine or to calculate a size of the dose of the medicament, which is set or dispensed by the injection device, on the basis of the sensor signal and on the basis of data received from one of the electronic container identifier and the electronic device identifier.

30. An injection system, comprising an injection device for injecting of a dose of a medicament, the injection device comprising:
(i) a housing comprising a body detachably connectable to a container part, wherein the container part is configured to accommodate a medicament container,
(ii) a drive mechanism arranged in the body and operable to expel the dose of the medicament from the medicament container, (iii) a first antenna on or in a first portion of the body, which is configured for wireless signal transmission with an electronic container identifier provided on or in the container part,
(iv) a second antenna on or in a second portion of the body electrically connected to the first antenna and configured for wireless signal transmission with a transceiver of an electronic module of an add-on device connectable to the injection device,
(v) an electronic device identifier on or inside the body and configured for wireless signal transmission with the transceiver, and an add-on device for attaching to the injection device, the add-on device comprises:
(i) a device body fastenable to a portion of the injection device,
(ii) an electronic module comprising a module processor and a transceiver, wherein the electronic module is operable:
(iii) to read data from the electronic container identifier via the first antenna and the second antenna, and
(iv) to read data from the electronic device identifier wirelessly.

31. The injection system according to claim 30, wherein the electronic device identifier of the injection device comprises an integrated device circuit with a device memory configured to store at least one of a device information and a use-related data.

32. The injection system according to claim 30, wherein the container memory is readable by the transceiver of the electronic module of the add-on device, through wireless signal transmission between the first antenna and the container antenna and through wireless signal transmission between the second antenna and the transceiver.

33. The injection system according to claim 30, wherein the transceiver is configured for wireless signal transmission with the second antenna when the device body is fastened to the injection device.

34. The injection system according to claim 30, wherein the transceiver is configured for wireless signal transmission with an identifier antenna of the electronic device identifier when the device body is fastened to the injection device.

35. The injection system according to claim 30, wherein the electronic module is operable to separate and/or to distinguish between signals received from the electronic container identifier and the electronic device identifier.
